(19) United States
(12) Patent Application Publication
(10) Pub. No.: US 2008/0029091 A1
(43) Pub. Date: Feb. 7, 2008

(54) Method and Device for Administering Xenon to Patients

(76) Inventor: Rainer Mullner, Wiener Neudorf (AT)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/594,025

(22) PCT Filed: Mar. 24, 2005
(86) PCT No.: PCT/EP05/03169

§ 371 Date: Aug. 10, 2007

(30) Foreign Application Priority Data
Mar. 26, 2004 (DE) 10 2004 015 406.6

(51) Int. Cl. A61M 16/01; A61K 33/00; A61M 16/10

(52) U.S. Cl. 128/203.12; 424/600

(57) ABSTRACT

The invention relates to a device and a method for administering xenon and/or a xenon-containing medium, especially a xenon-containing gas mixture, to a patient, whereby said patient is connected to an inhalation cycle and to a cardio-pulmonary bypass cycle (CPB cycle). According to the invention, the device comprises a) at least one source (X) for xenon and/or for a xenon-containing medium, b) at least one supply unit for xenon and/or for a xenon-containing medium to the inhalation cycle (V, 1, 1′) and to the CPB cycle (M, 2, 2′), c) at least one metering unit (S) for administering the xenon and/or a xenon-containing medium to the inhalation cycle (V, 1, 1′) and to the CPB cycle, and d) at least one analysis unit (S) for determining the xenon-content in the inhalation cycle (V, 1, 1′) and/or the CPB cycle (M, 2, 2′).

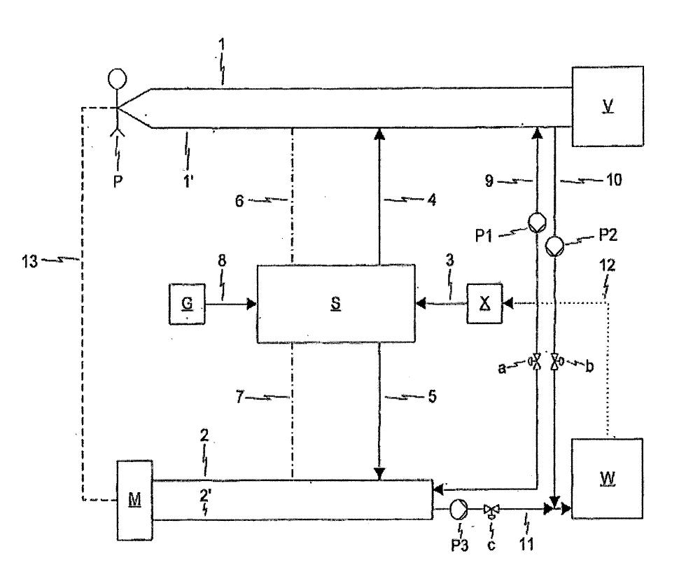

The invention relates to a device as well as to a process for administering xenon and/or a xenon-containing medium, in particular a xenon-containing gas mixture, to a patient, whereby the patient is connected to an inhalation system and a cardio-pulmonary-bypass system (CPB system).

The term “administration” is defined below, as always, as the application, administration, supply, etc., of xenon or a xenon-containing medium to (a) patient(s) in any way or form whatsoever.

Xenon is a rare noble gas that occurs in the earth's atmosphere. Its recovery—by means of cryogenic air separation—is comparatively intensive and costly. Xenon has both a demonstrated anesthetic action and a protective action against neurotoxic events. For example, in an open-heart operation, there is a comparatively great danger that a neurotoxic event will occur. In such operations, the cardio-pulmonary (blood) system in the body is bypassed, and the patient is supplied with oxygen via an external membrane, a so-called oxygenator. This application is also referred to as a cardio-pulmonary bypass (CPB).

Normally, an open-heart operation is accomplished as described below. First, artificial respiration is administered to the patient via a ventilator and an anesthesia machine. In this phase, the tissue of the patient may already be flooded with xenon. Then, the cardiopulmonary system is bypassed, and the oxygenator is connected. In this phase, xenon can pass into the tissue of the patient via the previously-mentioned oxygenator. Normally, the ventilator is or can be turned off during the operation of the CPB system. After the operation is completed, it is switched from the CPB system again to the ventilator or to the inhalation system. Also in this phase, the patient can be supplied preferably with xenon—via the inhalation system.

Since, however, as already mentioned, xenon is very expensive, an attempt is made to carry out the previously-described procedure in a manner that makes it possible to keep the consumption of xenon as low as possible.

The object of this invention is to indicate a generic device as well as a generic process for the administration of xenon and/or a xenon-containing medium, in particular a xenon-containing gas mixture, to a patient by means of which a controlled addition of xenon or a xenon-containing medium in the inhalation system and/or CPB system can be carried out.

To achieve this object, a generic device is proposed that

a) comprises at least one source of xenon and/or of a xenon-containing medium,

b) at least one supply unit for xenon and/or for a xenon-containing medium in the inhalation system and in the CPB system,

c) at least one dosage unit for the administration of xenon and/or a xenon-containing medium in the inhalation system and in the CPB system, and

d) at least one analysis unit for determining the xenon content in the inhalation system and/or the CPB system.

The process according to the invention is characterized in that

a) the xenon content in the inhalation system and/or the CPB system is determined directly or indirectly, and

b) xenon and/or a xenon-containing medium from a source of xenon and/or of a xenon-containing medium is administered at least occasionally in the inhalation system and/or in the CPB system.

The administration of xenon and/or a xenon-containing medium can be carried out via a regulator algorithm by the measured xenon concentration being compared to the set xenon concentration and the metering of xenon and/or a xenon-containing medium being carried out based on the difference of the two concentration values.

In addition to this regulator algorithm, preferably a safety mechanism or program is provided that prevents the xenon from being administered in an undesirable or harmful overdosage into the inhalation system and/or CPB system or from dropping below a desired or necessary O₂ concentration. Thus, for example, the minimum O₂ concentration can be set at 20%, by which an under-supply of O₂ (hypoxia) can be prevented.

As an alternative to this, however, a manual administration of xenon and/or a xenon-containing medium can also be carried out; in this case, constant xenon and O₂ flows are set. In this case, the measurement of the xenon concentration is used only for monitoring.

In principle, the xenon concentration in the inhalation line or before the oxygenator and/or in the exhalation line or after the oxygenator can be determined.

The device according to the invention, the process according to the invention, as well as additional embodiments thereof, which represent the subjects of the dependent claims, are explained in more detail below based on the diagrams depicted in the FIGURE, which shows a preferred embodiment of this invention.

Below:

P means patient

V means ventilator/anesthesia device

S means control, metering and supply unit

X means source of xenon and/or of a xenon-containing medium

G means a source for a one- or multi-component gas or gas mixture

M means a membrane of the CPB system

W means a xenon-reprocessing or xenon-recovery unit

a, b and c mean regulating valves

P1-P3 mean pumps

1-5/8-12 mean gas or medium lines

6 and 7 mean measuring lines

13 means a hose system.

As depicted in the FIGURE, artificial respiration is administered to a patient P by means of an anesthesia device V via a line 1. The respiratory gas stream that is exhaled by the patient P is fed back to the anesthesia device V via the line 1′. Also, the patient P is connected via a membrane M—depicted by the line 13 that is shown in dotted lines and that symbolizes a hose system via which the blood of the patient is pumped through the oxygenator—to a CPB system, depicted by the lines 2 and 2′.

The CPB system consists of, for example, a pump, which repeatedly supplies the gas already guided through the oxygenator to the oxygenator, by which a closed system is produced. Since the gas (mixture) that leaves the oxygenator is in most cases enriched with CO₂, the gas (mixture), before it is fed back to the oxygenator, is preferably guided through a CO₂-absorber and/or adsorber, not depicted in the FIGURE, and thus reduces the CO₂ concentration.

Via a central control unit S, in which the necessary analysis and metering functions are implemented, the xenon contents in the inhalation system 1 and 1′ as well as the CPB system 2 and 2′ are determined via the measuring lines 6 and 7. To keep the xenon consumption low, the analysis gas stream(s) is or are fed back to the inhalation system or the CPB system.

By means of the measuring lines 6 and 7, moreover, additional parameters of the inhalation system and/or CPB system, which are detected by means of corresponding measuring devices, can advantageously be forwarded to the control unit S. Such parameters are, for example, the concentrations of additional gas (mixture)s, flows, pressures, temperatures, etc.

The control unit S is connected via the line 3 to a source X of xenon and/or a xenon-containing medium, in particular a xenon-containing gas mixture. In this connection, the source X of xenon and/or a xenon-containing medium can comprise suitable storage units for xenon and/or a xenon-containing medium in gaseous form, in liquid form and/or in the form of a solution, for example a common salt solution. The xenon can also be present in the form of a donor of xenon, whereby the donor comprises a gas (mixture), a liquid, a solid or a solution.

The previously mentioned control unit S can be associated in addition—as depicted in the FIGURE—via line 8 with at least one additional source G of a one- or multi-component gas or gas mixture. In this connection, the source G is used in particular for the storage and dispensing of gas mixtures, such as air, oxygen, carbon dioxide, nitrogen oxide, anesthesia gases, volatile anesthesias, etc. For the possible embodiments of source G, the statements made with respect to the source X hold true.

Based on the determined xenon content(s) in the inhalation system and/or the CPB system, xenon or a xenon-containing medium in the desired concentration can be added in measured quantities to the latter via the lines 4 and/or 5. In this connection, this addition of measured quantities in the system or the systems can take place either simultaneously or at other times.

A medium exchange between the inhalation system and the CPB system can be carried out via the line 9 that connects the two systems to one another and in which a regulating valve a and optionally a pump P1 are arranged. In particular then, use is made of this possibility if the gas is no longer used in one of the systems. Thus, for example, after the CPB treatment is completed, the gas (mixture) can be diverted into the inhalation system.

In addition, the inhalation system and the CPB system can be connected to a recovery unit or a reprocessing unit W via the lines 10 or 11, in which regulating valves b or c and optionally pumps P2 or P3 are also arranged. The latter unit is used in the recovery and optionally reprocessing of xenon from the gas or fluid mixtures of the inhalation system and/or CPB system. In this connection, the xenon recovery is carried out by means of suitable measures, such as, for example, filtering, absorption, adsorption, compression, etc.

The xenon that is obtained in the recovery unit or reprocessing unit W is normally collected and reprocessed at the plant. Assuming a corresponding embodiment of the reprocessing unit—the direct supply of the reprocessed xenon via the line 12, shown in dotted lines, to the xenon source X would also be conceivable, however.

The invention makes it possible to add xenon or a xenon-containing medium in measured quantities to the inhalation system and/or to the CPB system at the same time or at other times. In this connection, the addition of measured quantities to one or to both systems can be program-controlled. This program control makes it possible that the administration of xenon or a xenon-containing medium can be carried out at an optimum time.

Thus, for example, the administration of xenon or a xenon-containing medium as well as the administration, optionally to be provided, of additional gas (mixture)s can be carried out exclusively before the CPB treatment, exclusively during the CPB treatment or exclusively after the CPB treatment. In addition, almost any combinations, such as, for example, before and during the CPB treatment, are possible.

As already mentioned, preferably a safety mechanism or safety program is provided that prevents xenon from being administered in an undesirable or harmful overdosage into the inhalation system and/or CPB system or from dropping below a desired or necessary O₂ concentration.

If, for example, air is found in the inhalation system—consequently an oxygen concentration of about 21% exists—and xenon is fed to the inhalation system in an amount that establishes a xenon concentration of 60%, the air concentration within the inhalation system thus is only 40%. This had the result that the oxygen concentration dropped to about 8.4%, which caused the patient to suffer from an under-supply of oxygen (hypoxia).

By means of such a safety program, in addition it can be achieved that the administration of xenon or a xenon-containing medium is possible only starting from a presettable or preset oxygen concentration value. If this oxygen concentration value is set at 90%, for example, and xenon is fed to the system in an amount such that a xenon concentration of 60% is established, the remaining 40% in the system consists of 90% oxygen. Consequently, the oxygen concentration within the system was an uncritical 36%.

By means of the safety program, moreover, it can be ensured that before the administration of xenon or a xenon-containing medium, flushing with oxygen is carried out, by which a sufficiently high and thus also sufficiently reliable oxygen concentration can be ensured.

In addition, it is advantageous that both systems can be supplied from a common source of xenon or of the xenon-containing medium. Moreover, unconsumed xenon can be recovered from both systems, and/or xenon, which is not (no longer) required in one of the two systems, can be fed to the respective other system.

1. Device for administering xenon and/or a xenon-containing medium, in particular a xenon-containing gas mixture, to a patient, whereby the patient is connected to an inhalation system and to a cardio-pulmonary bypass system (CPB system), characterized in that the device comprises
a) at least one source (X) of xenon and/or of a xenon-containing medium,
b) at least one supply unit for xenon and/or for a xenon-containing medium in the inhalation system (V, 1, 1′) and in the CPB system (M, 2, 2′),
c) at least one dosage unit (S) for administering xenon and/or a xenon-containing medium in the inhalation system (V, 1, 1′) and in the CPB system, and
d) at least one analysis unit (S) for determining the xenon content in the inhalation system (V, 1, 1′) and/or the CPB system (M, 2, 2′).

2. Device according to claim 1, wherein the source (X) of xenon or of a xenon-containing medium is a source that yields gaseous xenon, optionally in a mixture with one or more other media, preferably gases.

3. Device according to claim 1, wherein means for connecting the inhalation system (V, 1, 1′) and the CPB system (M, 2, 2′), via which a media exchange (9) can be carried out between the two systems, are provided.

4. Device according to claim 1, wherein at least one reprocessing unit (W), which is connected to or can be connected to the inhalation system (V, 1, 1′) and/or the CPB system (M, 2, 2′) and that is used for the recovery of xenon from the previously mentioned system or system, is provided.

5. Device according to claim 1, wherein in addition to the analysis unit (S) for determining the xenon content in the inhalation system (V, 1, 1′) and/or the CPB system (M, 2, 2′), at least one additional analysis unit, which is used to determine a media concentration and/or another parameter, such as flow, pressure, temperature, etc., is provided.

6. Device according to claim 1, wherein the CPB system (M, 2, 2′) is designed as a closed system.

7. Device according to claim 1, wherein the CPB system (M, 2, 2′) has a CO₂ absorber, a CO₂ adsorber and/or a CO₂ filtering device, preferably a permeative CO₂ filtering device.

8. Process for the administration of xenon and/or a xenon-containing medium, in particular a xenon-containing gas mixture, to a patient, whereby the patient is connected to an inhalation system and a cardio-pulmonary bypass system (CPB system), wherein
the xenon content in the inhalation system (V, 1, 1′) and/or the CPB system (M, 2, 2′) is determined directly or indirectly, and
xenon and/or a xenon-containing medium is at least occasionally administered from a source (X) of xenon and/or of a xenon-containing medium in the inhalation system (V, 1, 1′) and/or in the CPB system (M, 2, 2′).

9. Process according to claim 8, wherein at least one additional medium, preferably a gas or gas mixture, is at least occasionally fed to the inhalation system (V, 1, 1′) and/or to the CPB system (M, 2, 2′).

10. Process according to claim 8, wherein in addition to the xenon content(s), additional media concentrations and/or parameters, such as flow, pressure, temperature, etc., of the inhalation system (V, 1, 1′) and/or the CPB system (M, 2, 2′), are detected.

11. Process according to claim 8, wherein the unconsumed xenon, contained in the inhalation system (V, 1, 1′) and/or the CPB system (M, 2, 2′), is recovered (W).

12. Process according to claim 8, wherein in the administration of xenon and/or a xenon-containing medium in the inhalation system (V, 1, 1′) and/or the CPB system (M, 2, 2′), the values in question cannot drop below a preset or presettable oxygen concentration in the inhalation system (V, 1, 1′) and/or the CPB system (M, 2, 2′).
